(19) United States
(12) Patent Application Publication
(10) Pub. No.: US 2026/0130646 A1
(43) Pub. Date: May 14, 2026

(54) ULTRASOUND TRANSDUCER FOR IVUS CATHETERS

(71) Applicant: Boston Scientific Scimed, Inc., Maple Grove, MN (US)

(72) Inventors: Marshall Lohrenz, Minnetonka, MN (US); Robert Fagerlund, White Bear Lake, MN (US); Chad Brandvold, Wyoming, MN (US); Jaymes Olson, Lino Lakes, MN (US); Andrew Brian Graveley, Shoreview, MN (US); John Ledder, New Brighton, MN (US)

(73) Assignee: Boston Scientific Scimed, Inc., Maple Grove, MN (US)

(21) Appl. No.: 19/382,492

(22) Filed: Nov. 7, 2025

Related U.S. Application Data
(60) Provisional application No. 63/718,057, filed on Nov. 8, 2024.

(51) Int. Cl. A61B 8/00; A61B 8/12; B06B 1/06; H10N 30/088

(52) U.S. Cl. CPC A61B 8/445; A61B 8/12; B06B 1/067; H10N 30/088; B06B 2201/76

(57) ABSTRACT

Methods of manufacturing transducers for intravascular ultrasound catheters. An illustrative method may comprise forming a composite PZT structure, applying a backing layer, applying a metallic layered coating to the backing layer dicing the composite PZT structure and backing layer to form an array of transducer elements, wherein at least some transducer elements remain interconnected at the backing layer after dicing, applying a matching layer, and creating a connection point through the matching layer.

### CROSS-REFERENCE TO RELATED APPLICATIONS

This application claims the benefit of priority under 35 U.S.C. § 119 of U.S. Provisional Application No. 63/718,057, filed Nov. 8, 2024, the entire disclosure of which is hereby incorporated by reference.

### TECHNICAL FIELD

The present disclosure pertains to medical imaging as well as systems and methods for medical imaging. More particularly, the present disclosure pertains to ultrasound transducers and methods of making ultrasound transducers.

### BACKGROUND

A wide variety of medical imaging systems and methods have been developed for medical use, for example, use in imaging vascular anatomy. Some of these systems and methods include intravascular imaging modalities. These systems and methods include various configurations and may operate or be used according to any one of a variety of methods. Of the known vascular imaging systems and methods, each has certain advantages and disadvantages. Accordingly, there is an ongoing need to provide alternative systems and methods for vascular imaging and assessment.

### BRIEF SUMMARY

This disclosure provides design, material, manufacturing method, and use alternatives for medical devices. An intravascular imaging system and components thereof are disclosed.

In an example, a method of manufacturing an ultrasound transducer for use in an intravascular ultrasound catheter may include forming a composite PZT structure, applying a backing layer to the composite PZT structure, applying a metallic layered coating to the backing layer, dicing the composite PZT structure and the backing layer to form an array of transducer elements, wherein two or more transducer elements might remain mechanically connected at the backing layer after dicing, applying a matching layer to the diced composite PZT structure, and creating a connection point through the matching layer.

Alternatively or additionally to any of the examples above, in another example, dicing the composite PZT structure and the backing layer may include performing a first dicing operation to separate the array of transducer elements into columns, and performing a second dicing operation to partially separate the array of transducer elements into rows while maintaining electrical connectivity between transducer elements through the backing layer.

Alternatively or additionally to any of the examples above, in another example, dicing the composite PZT structure and the backing layer may include performing a first dicing operation to partially separate the array of transducer elements into columns while maintaining electrical connectivity between transducer elements through the backing layer, and performing a second dicing operation to separate the array of transducer elements into rows.

Alternatively or additionally to any of the examples above, in another example, dicing the composite PZT structure and the backing layer may include performing a first dicing operation to partially separate the array of transducer elements into columns while maintaining electrical connectivity between transducer elements through the backing layer, and performing a second dicing operation to partially separate the array of transducer elements into rows while maintaining electrical connectivity between transducer elements through the backing layer.

Alternatively or additionally to any of the examples above, in another example, the method may further include testing the transducer elements while they remain connected in the array of transducer elements.

Alternatively or additionally to any of the examples above, in another example, testing the transducer elements may include coupling a negative lead to a backing layer of a first transducer element in a column of the array of transducer elements, coupling a positive lead to a connection point on the first transducer element, measuring an electrical property of the first transducer element, maintaining the coupling of the negative lead to the backing layer, and sequentially coupling the positive lead to connection points on subsequent transducer elements in the column to measure the electrical property of each transducer element in the column.

Alternatively or additionally to any of the examples above, in another example, the method may further include singulating the transducer elements.

Alternatively or additionally to any of the examples above, in another example, forming the composite PZT structure may include dicing a first PZT wafer and a second PZT wafer, applying a layer of a polymer coating to the diced surfaces of the PZT wafers, and pressing the first and second diced PZT wafers together to form the composite PZT structure.

Alternatively or additionally to any of the examples above, in another example, after dicing the first PZT wafer and the second PZT wafer, each of the first and second PZT wafers may include a plurality of pillars.

Alternatively or additionally to any of the examples above, in another example, each pillar of the plurality of pillars may have a width to height ratio in the range of 0.15 to about 0.35.

Alternatively or additionally to any of the examples above, in another example, applying the backing layer may include casting a conductive epoxy onto the composite PZT structure, curing the conductive epoxy, and grinding the cured conductive epoxy to a specified thickness.

Alternatively or additionally to any of the examples above, in another example, applying a matching layer to the diced composite PZT structure may include depositing a conductive deposit on the diced composite PZT structure, and applying the matching layer over the conductive deposit and the diced composite PZT structure, and wherein creating a connection point through the matching layer may include removing a portion of the matching layer to expose the conductive deposit.

In an example, an ultrasound transducer for an intravascular ultrasound catheter may include a backing layer having a lower portion and an upper portion, a piezoelectric layer disposed on the upper portion of the backing layer, a matching layer disposed on the piezoelectric layer, and a connection point formed through the matching layer, wherein the lower portion of the backing layer might have a first length greater than a second length of the upper portion of the backing layer.

Alternatively or additionally to any of the examples above, in another example, the lower portion of the backing layer may extend beyond the upper portion to form a ledge.

Alternatively or additionally to any of the examples above, in another example, the ledge may extend around an entire perimeter of the backing layer.

Alternatively or additionally to any of the examples above, in another example, the ultrasound transducer may further include a pre-tin solder disposed on the piezoelectric layer at the connection point.

Alternatively or additionally to any of the examples above, in another example, the backing layer may include a conductive epoxy.

Alternatively or additionally to any of the examples above, in another example, the ultrasound transducer may further include a metallic layered coating on a bottom surface of the backing layer.

Alternatively or additionally to any of the examples above, in another example, the matching layer may extend along a lateral and/or axial surface of the piezoelectric layer.

Alternatively or additionally to any of the examples above, in another example, the matching layer may extend along at least a portion of a lateral and/or axial surface of the backing layer.

In an example, an ultrasound transducer for an intravascular ultrasound catheter may include a backing layer having a lower portion, an intermediate portion, and an upper portion, a piezoelectric layer disposed on the upper portion of the backing layer, a matching layer disposed on the piezoelectric layer, and a connection point formed through the matching layer, wherein the lower portion of the backing layer might have a first length, the intermediate portion might have a second length less than the first length, and the upper portion might have a third length less than the second length.

Alternatively or additionally to any of the examples above, in another example, the intermediate portion of the backing layer may extend proximally beyond a proximal end of the upper portion to form a first ledge.

Alternatively or additionally to any of the examples above, in another example, the first ledge may extend proximally to a proximal end of the piezoelectric layer.

Alternatively or additionally to any of the examples above, in another example, the lower portion of the backing layer may extend proximally beyond a proximal end of the intermediate portion to form a second ledge.

Alternatively or additionally to any of the examples above, in another example, the lower portion of the backing layer may extend distally beyond a distal end of the intermediate portion to form a third ledge.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device;

FIG. 2 is a side view of a distal end region of an example imaging assembly;

FIG. 3 is an illustrative flow diagram of an illustrative method for forming one or more transducers;

FIG. 4 is a perspective view of a portion of a partially assembled illustrative composite PZT wafer;

FIG. 5 is a perspective view of an illustrative array of a plurality of transducers on a dicing tape;

FIG. 6A is a top view of a portion of the array of transducers of FIG. 5;

FIG. 6B is a perspective view of a portion of the array of transducers of FIG. 5;

FIG. 7 is a perspective view of an illustrative transducer;

FIG. 8 is a perspective view of the illustrative transducer of FIG. 7 with a pre-tin solder;

FIG. 9 is a perspective view of another illustrative transducer;

FIG. 10 is a perspective view of another illustrative transducer; and

FIG. 11 is a perspective view of another illustrative transducer.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, or characteristics. However, such recitations do not necessarily mean that all embodiments include the particular features, structures, or characteristics. Additionally, when particular features, structures, or characteristics are described in connection with one embodiment, it should be understood that such features, structures, or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Rotational intravascular ultrasound (IVUS) may be used for high-resolution imaging of the blood vessels during percutaneous coronary interventions (PCI). Increasing adoption of intravascular imaging for PCI may increase production volumes of IVUS catheters. Current IVUS catheters may require hand-assembly of microscopic components. This manual approach may be highly dependent on operator technique which may significantly impact production yields. The present disclosure is directed towards distal tip designs to increase manufacturability while maintaining device performance. While the present disclosure is described with respect to intravascular imaging, the devices and methods described herein can be used for pulmonary procedures/imaging or in other anatomy, as desired.

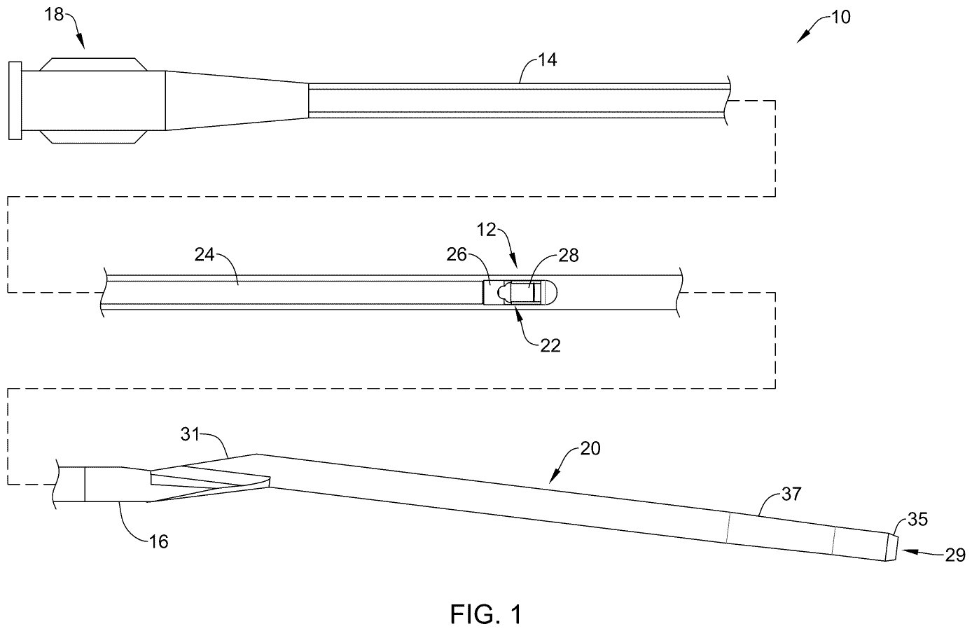

FIG. 1 is a side view of an example medical device 10. In at least some instances, the medical device 10 takes the form of an imaging medical device. For example, the medical device 10 may be an IVUS device that may be used to image a blood vessel. The structure/form of the medical device 10 can vary. In some instances, the medical device 10 may include an elongate shaft 12 having a proximal end region 14 and a distal end region 16. A proximal hub or connector 18 may be coupled to or otherwise disposed adjacent to the proximal end region 14 of the elongate shaft 12. A tip member 20 may be coupled to or otherwise disposed adjacent to the distal end region 16 of the elongate shaft 12. The tip member 20 may include a guidewire lumen 29 having a guidewire exit port 31, an atraumatic distal end 35, one or more radiopaque markers 37, and/or other features. In some embodiments, the tip member 20 may extend at a non-parallel angle to the proximal end region 14 of the elongate shaft 12. An imaging assembly 22 may be movably disposed within a lumen of the shaft 12. In general, the imaging assembly may be used to capture/generate images of a blood vessel. In some instances, the medical device may include devices and/or features similar to those disclosed in U.S. Patent Application Pub. No. 2012/0059241 and U.S. Patent Application Pub. No. 2017/0164925, the entire disclosures of which are herein incorporated by reference. In at least some instances, the medical device 10 may resemble and/or include features that resemble the OPTICROSS™ Imaging Catheter or the OPTICROSS™ HD Imaging Catheter, commercially available from BOSTON SCIENTIFIC, Marlborough, MA.

The imaging assembly 22 may include a drive cable or shaft 24, a housing 26, and an imaging member or transducer 28 coupled to the drive cable 24 and/or housing 26. In at least some instances, the transducer 28 includes an ultrasound transducer. Other transducers are also contemplated. The transducer 28 may be rotatable and/or axially translatable relative to the shaft 12. For example, the drive cable 24 may be rotated and/or translated in order to rotate and/or translate the transducer 28 (and the housing 26).

While not explicitly shown, the medical device 10 may include a telescoping section, configured to allow the medical device operator to move the drive shaft 24 including the imaging assembly 22 proximally and distally within the elongate shaft 12, without having to move the entire elongate shaft 12 within the patient. This allows the catheter operator to easily change the location of the imaging assembly 22 or another medical device within the patient. For example, the telescoping section may be actuated to change the location of the imaging assembly 22 within the elongate shaft 12. An illustrative telescoping section is described in commonly assigned U.S. Patent Application Pub. No. 2023/0309962, the disclosure of which is herein incorporated by reference.

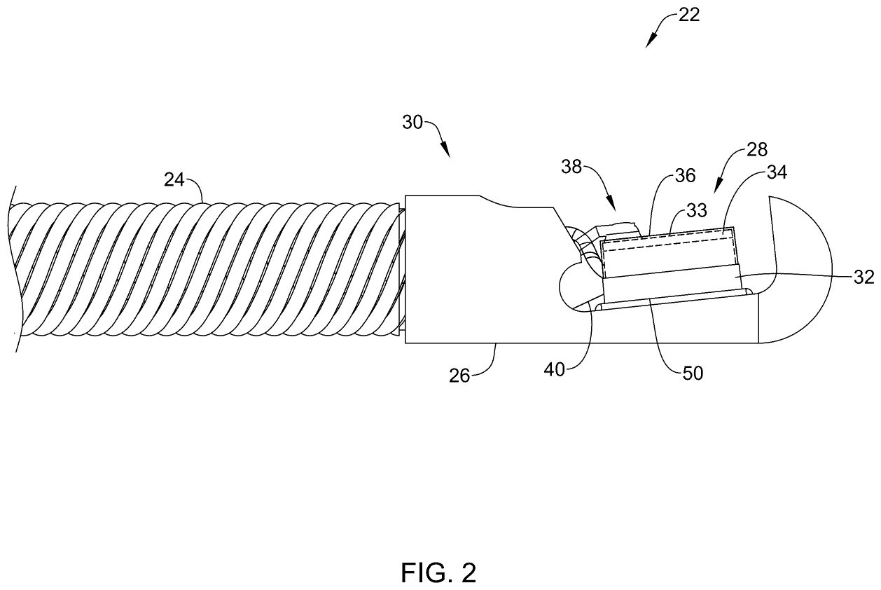

FIG. 2 is a side view of a distal end region 30 of the imaging assembly 22. The transducer 28 may be configured to allow for an automated pick-and-place machine to assemble the transducer 28 into the housing 26. Further, electrical connections may be made by automatic dispensing of an electrically conductive adhesive (ECA) or solder. The housing 26 may include features which facilitate automated pick-and-place assembly which are described in commonly assigned patent application attorney docket number 2001.3746100 titled “Distal Tip for IVUS Catheters” filed of even date herewith, which is hereby incorporated by reference. While the present structure increases the manufacturability and/or assembly of the distal end region 30 of the imaging assembly 22, the performance of the imaging assembly 22 is not compromised.

The transducer 28 may include a backing layer 32, a layer with piezoelectric properties 34, such as, but not limited to, a lead zirconate titanate (PZT) layer, and a matching layer 36. As will be described in more detail herein, the matching layer 36 may extend across an upper surface 33 and optionally along the lateral sides of the piezoelectric layer 34 as well as at least a portion of the lateral sides of the backing layer 32. The transducer 28 may be electrically coupled to an imaging system (not explicitly shown) through a first electrical connection 38 and a second electrical connection 40.

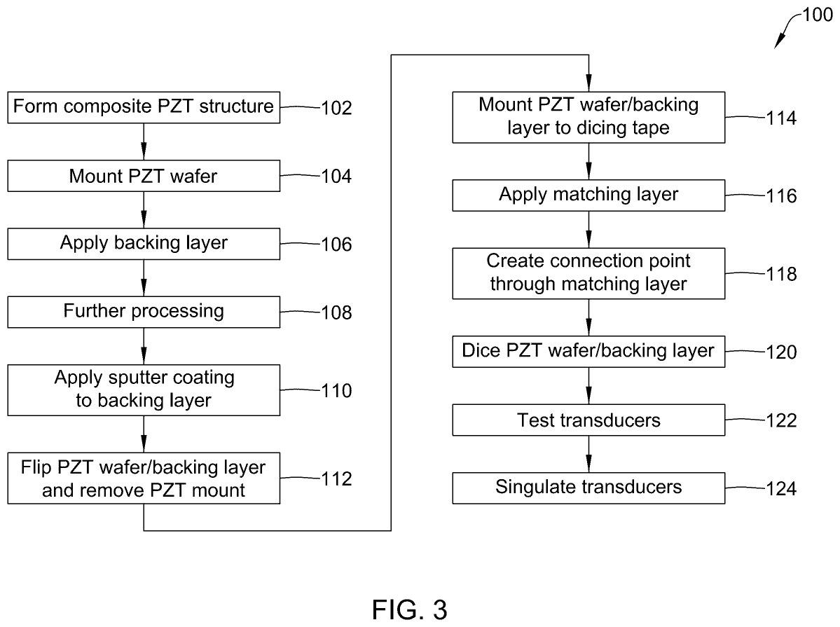

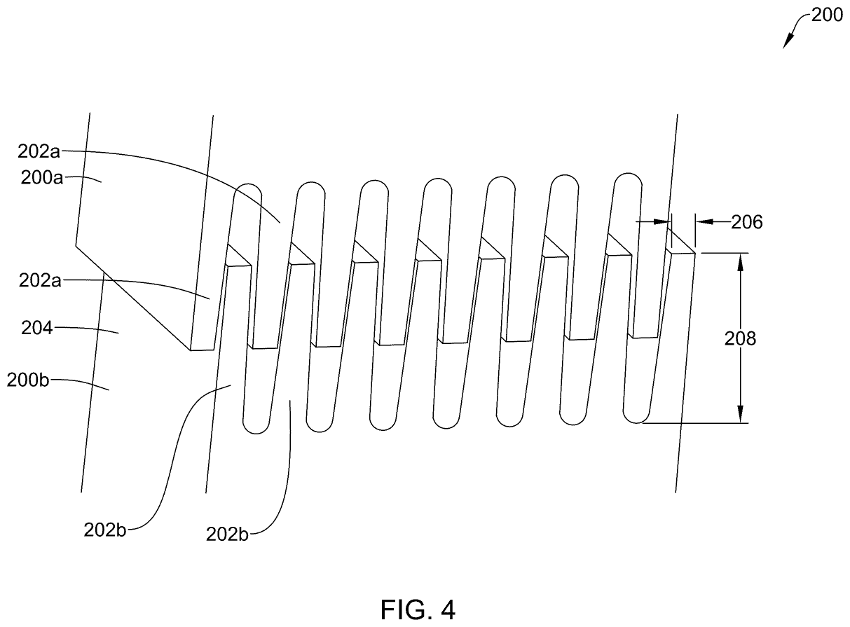

FIG. 3 is an illustrative flow diagram 100 of an illustrative method for forming one or more transducers 28. While certain steps are shown or described as a sequence, in other embodiments fewer steps are contemplated and the order by which steps are performed can be different than what is illustrated. Generally, a plurality of transducers 28 may be formed from a single block having a length and width greater than a length and width of the transducer 28. To begin, a composite PZT wafer or structure may be formed, as shown at block 102. In some embodiments, the PZT wafer may be a generally solid PZT wafer. The PZT wafer may have a length and width that is greater than the length and width of the transducer 28 such that the PZT wafer may be used to manufacture a plurality of transducers 28. Generally, a composite PZT wafer may be formed by mating two diced PZT wafers with an epoxy disposed therebetween. In another example, a thin layer of a polymer coating (i.e., parylene) may be added to the diced PZT surface prior to mating the diced PZT wafers to allow the two wafers to be thermally bonded together. The PZT wafers may be heated and pressed together to bond the two wafers which may provide a reduced profile composite wafer which is easier to process. Referring briefly to FIG. 4, which illustrates a perspective view of a portion of a partially assembled illustrative composite PZT wafer 200, the composite wafer 200 may include a first diced PZT wafer 200a and a second diced PZT wafer 200b. The diced wafers 200a, 200b may each include a plurality of pillars 202a, 202b (for brevity and ease of understanding, every pillar 202a, 202b is not identified with a reference number). The diced wafers 200a, 200b may be annealed prior to pressing the diced wafers 200a, 200b together. As the diced wafers 200a, 200b are pressed together (FIG. 4 illustrates the diced wafers 200a, 200b partially together), there may be a gap or kerf 204 (for brevity and ease of understanding, every gap is not identified with a reference number) between adjacent pillars 202a, 202b. The gaps 204 may be filled with a kerf filler, such as, but not limited to, an epoxy, parylene, or other adhesive. For example, an epoxy, parylene, or other adhesive is disposed over the diced surfaces prior to pressing the diced wafers 200a, 200b together. In some embodiments, the kerf filler may extend above the height 208 of the pillars 202a, 202b. The aspect ratio of the pillars (e.g., the width 206 to height 208 ratio) may impact the performance of the PZT wafer 200. For example, a squarer aspect ratio (e.g., in the range of about 0.5 to about 1) may decrease performance as the PZT wafer 200 may resonate in a lateral mode that is close in frequency to that of the thickness mode. Said differently, energy that could be used for imaging may be wasted on resonance in the lateral direction within the PZT wafer 200. In some embodiments, the diced wafers 200a, 200b may have a more rectangular (e.g., less square) width 206 to height 208 ratio (e.g., aspect ratio) in the range of about 0.15 to about 0.35. In one illustrative non-limiting example, the pillars 202a, 202b may have a height 208 of about 25-40 micrometers (μm), or about 30-35 μm, or about 33 μm and a width 206 of about 5-20 μm, or about 10-12 μm, or about 11 μm. This may help to reduce the aspect ratio, which may help to reduce wasted energy/resonance. However, other heights 208 and/or widths 206 may also be used. In some cases, the gaps 204 may have width that is similar to the width 206 of the pillars 202a, 202b. In other examples, the gaps 204 may have a width that is less than or about half the width 206 of the pillars 202a, 202b. The upper and lower surfaces of the PZT wafer 200 may be sputter coated to increase adhesion and/or improve electrical connections between the PZT wafer 200 and other layers of the transducer 28.

Returning to FIG. 3, once the PZT wafer 200 is constructed (composite or solid), the PZT wafer 200 may be optionally mounted to tape or a glass plate, as shown at block 104. Next, a backing layer (similar in form and function to backing layer 32) may be cast onto the PZT wafer 200, as shown at block 106. In some embodiments, the backing layer may be a conductive epoxy. Illustrative backing layers are described in commonly assigned patent application attorney docket number 2001.3750100 titled “Backing Layer for Intravascular Imaging Device” filed of even date herewith, which is hereby incorporated by reference. The backing layer may be further processed, as shown at block 108. It is contemplated that a grinding process may produce a superior electrical connection relative to a milled or lapped surface by not removing as much metal particles from the epoxy and creating a flatter, smoother surface. However, the surface of the backing layer may be milled or lapped if so desired.

A metallic layered coating, such as, but not limited to a sputter coating, (not explicitly shown in FIG. 2) may be coated on the bottom surface (e.g., surface opposite the surface contacting the PZT wafer 200) of the backing layer, as shown at block 110. The metallic layered coating may enhance the electrical connection between the backing layer 32 and a securement member 50 (see, for example, FIG. 2). Further, the metallic layered coating may reduce or eliminate cross-linking between the backing layer 32 and a dicing tape used to manufacture the transducers 28. In some examples, the securement member 50 may be a solder bond. It is contemplated that a thickness of the sputter layer may be increased to improve reliability of the coupling between the securement member 50 and the transducer 28. In some examples, the metallic layered coating may provide a low resistance electrical connection between individual transducers 28 during manufacture (e.g., before singulation) for electrical testing.

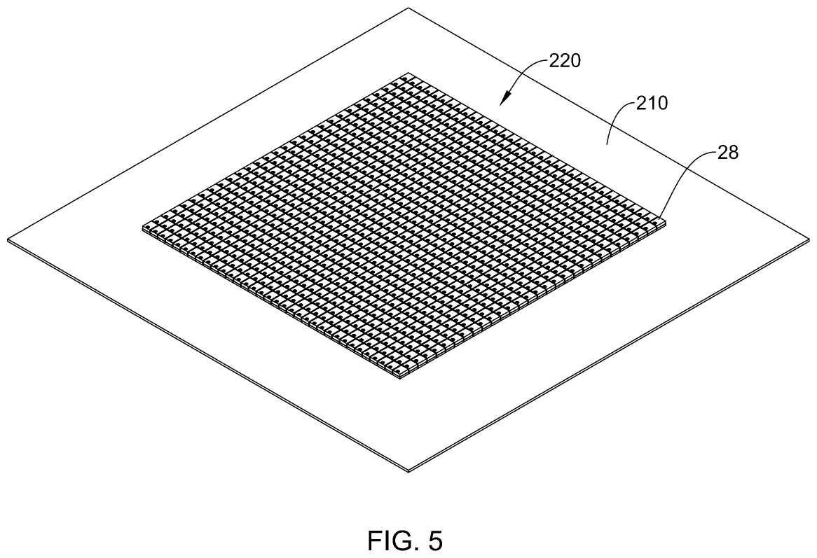

Next, the PZT wafer 200 and backing layer assembly may be flipped and the optional mount removed from the PZT wafer 200, as shown at block 112. The wafer 200 and backing layer assembly or block may be mounted to a dicing tape 210 (see, for example, FIG. 5), as shown at block 114.

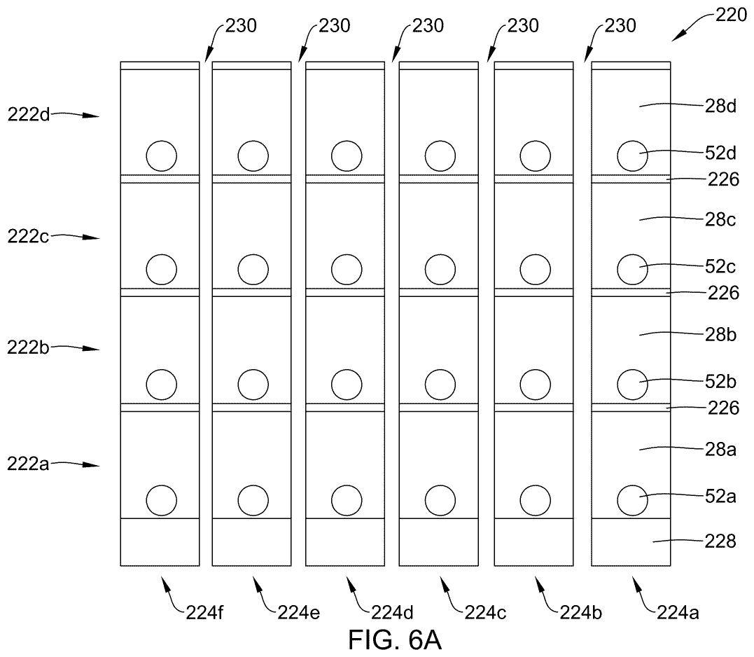

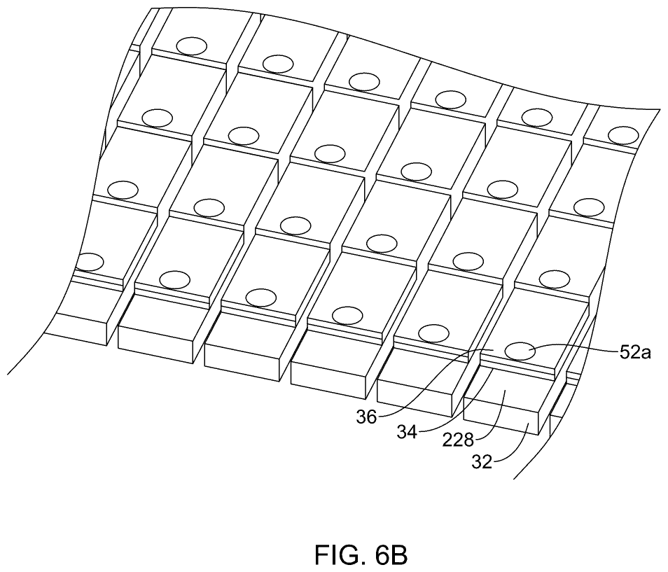

Next, a matching layer (similar in form and function to matching layer 36) may be applied to the surface of the PZT layer, as shown at block 116. In some embodiments, the matching layer may be applied to only the PZT layer. In other embodiments, the matching layer may extend along lateral sides of the backing layer, as will be described in more detail herein. In some embodiments, a conductive connective region may be a conductive deposit applied, deposited, or pre-tinned to the PZT layer prior to applying the matching layer, as will be described in more detail herein. A region of the matching layer may be removed or ablated to provide a connection point 52 (see, for example FIGS. 6A and 6B) for a first electrical connection 38 (FIG. 2) connecting a positive lead to the upper surface of the PZT layer, as shown at block 118. The connection point 52 may be generally circular or may take other shapes, such as, but not limited to square, rectangular, oblong, or the like. FIG. 5 is a perspective view of an illustrative array 220 of a plurality of transducers 28 on a dicing tape 210 formed by the preceding steps. It is contemplated that the number of transducers 28 in the array 220 may depend on the size of the PZT wafer 200 and/or the desired size of the transducers 28.

A dicing saw having a first blade thickness may be used to dice or cut the PZT wafer 200 and backing layer assembly into an array of transducer elements including a plurality of rows and a plurality of columns, as shown at block 120. The dice or cut may extend partially through an entire thickness of the backing layer such that at least some regions of the array remain at least partially mechanically and electrically connected at the backing layer. Said differently, two or more of the transducer elements of the array of transducer elements remain mechanically and electrically connected at the backing layer as the dicing cut does not extend though the entire thickness of the backing layer. The number of mechanically and electrically connected transducer elements may depend on the size of the array. In some cases, ten or more, twenty or more, thirty or more transducer elements may be electrically and mechanically connected after the first or initial dicing step 120. These are just some examples. The number of connected transducer elements may be less than ten or greater than thirty, as desired. In some embodiments, the rows of the array of transducer elements may be separated (e.g., the cut extends through an entire thickness of the PZT wafer 200/backing layer block) while the columns remain mechanically and electrically connected through the backing layer and metallic layered coating. and electrically connected at the backing layer after the initial or first dicing step 120.

Next, the transducers 28 may be electrically tested prior to singulation, as shown at block 122 in FIG. 3. FIG. 6A is a top view of a portion of the array 220 of transducers 28 (e.g., the transducers 28 are marked in FIG. 6A as transducers 28a-28d) and FIG. 6B is a perspective view of a portion of the array 220 of transducers 28. As described above, the array 220 may include a plurality of rows 222a-n, with four rows 222a-d shown in FIG. 6A, and a plurality of columns 224a-n, with six columns 224a-f shown in FIG. 6A. The array 220 may be diced such that that the transducers 28 forming the columns 224a-f are interconnected by a region 226 of backing layer and metallic layered coating while the transducers 28 forming the rows 222a-d are spaced from one another by a gap 230. Said differently, the transducers 28 forming the rows 222a-d may be free from connection at the backing layer and/or metallic layered coating. However, this is not required. The transducers 28 forming the columns 224a-f may be connected. The reverse configuration is also contemplated in which the transducers 28 forming the rows 222a-d are interconnected and the transducers 28 forming the columns 224a-f are free from connection.

In some embodiments, a ledge or step 228 or region of exposed backing layer may be present on at least one transducer 28a of the plurality of interconnected transducers 28a-d. To perform the test, a negative lead may be coupled with or placed in contact with the backing layer at the ledge or step 228 of a first column (e.g., column 224a) and a positive lead may be coupled to or placed in contact with the connection point 52a of a first transducer 28a. An electrical property, such as, but not limited to, impedance, may be measured to verify the electrical functioning of the transducer 28a. Other electrical properties may be used, as desired. To test the next transducer 28b, the negative lead may remain coupled to the ledge or step 228 as the backing layer and metallic layered coatings of the interconnected transducers 28a-d are physically connected and electrically connected. The positive lead may be coupled to or placed in contact with the connection point 52b of a second transducer 28b. Instead of needing to precisely place a probe onto the connection point 52a-d of each transducer 28a-d and the backing layer of each transducer 28a-d, it is only necessary to precisely place a probe on the ablation spot for each transducer while maintaining the negative lead at a single location for the entire column. Again, an electrical property, such as, but not limited to, impedance, may be measured to verify the electrical functioning of the transducer 28b. The positive lead may be repositioned to the connection point 52c-n of each remaining transducer 28c-d in the column 224a, and the process repeated for each transducer 28a-n in the column 224a. When testing has been performed on each transducer 28a-n of the first column 224a, the negative lead may be coupled to the backing layer or step of the first transducer of the next column and the process repeated. This may continue for each column 224a-n of the array 220. It is contemplated that minimizing the number of different connections to be completed may reduce the amount of time required to test a plurality of transducers 28.

After completion of the testing, the transducers 28 may be singulated, as shown at block 124 of FIG. 3. Singulation may be performed with a dicing saw. In some cases, the dicing saw may have a blade thickness that is similar to the blade thickness used to dice the array 220 into columns and rows. In other examples, the dicing saw may have a blade thickness that is thinner than the blade thickness used to dice the array 220 into columns and rows such that the transducers 28 include a region of backing layer having a first length greater than a second length adjacent to the PZT layer 34. Said differently, the dicing saw may remove less backing layer material during singulation than during the first dicing step (step 116), which may create axially (and/or laterally) extending edges or feet. During singulation, the ledge or step 228 used to perform the testing may be removed or the transducers 28 including the ledge or step 228 may be disposed of. In some embodiments, the transducers 28 may be singulated with a die-expansion method. After singulation, the transducers 28 may all be oriented the same, in a flat uniform array, fixed in place. This may make it easy to pick-and-place them manually or with an automated machine.

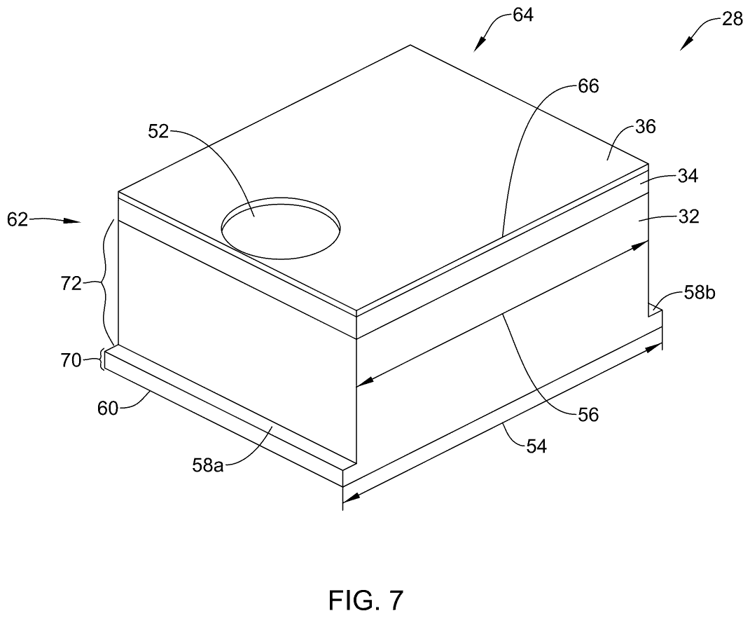

FIG. 7 is a perspective view of an illustrative transducer 28 formed by the process described with respect to FIG. 3. The transducer 28 includes a backing layer 32, a PZT layer 34, and a matching layer 36. A region of the matching layer 36 has been removed or ablated to define a connection point 52. The connection point 52 may be adjacent to a proximal end 62 of the transducer 28. The matching layer 36 may extend across an upper surface 66 of the PZT layer 34. However, in some cases, the matching layer 36 may extend along the lateral sides of the PZT layer 34 and/or portions of or an entirety of the lateral sides of the backing layer 32.

A lower portion 70 adjacent to a bottom surface 60 of the backing layer 32 may have first length 54 and an upper portion 72 adjacent to the PZT layer 34 may have a second length 56. The first length 54 may be greater than the second length 56. A longitudinal center of the region of the backing layer 32 having the first length 54 may be centered with a longitudinal center of the region of the backing layer 32 having the second length 56 to define a first longitudinally extending ledge or foot 58a adjacent to a proximal end 62 of the transducer 28 and extending along an entirety of a width thereof and a second longitudinally extending ledge or foot 58b adjacent to a distal end 64 of the transducer 28 and extending along an entirety of a width thereof. The thickness of the feet 58a, 58b may be determined during the initial dicing (step 116) The feet 58a, 58b may provide the electrical connection between adjacent transducers during the manufacturing process (e.g., when the transducers 28 are in an array 220). The feet 58a, 58b may be created when a dicing saw is used for singulation which has a thinner blade than the dicing saw used for initial dicing (e.g., step 116 of FIG. 3). For example, less material may be removed during singulation with a thinner blade.

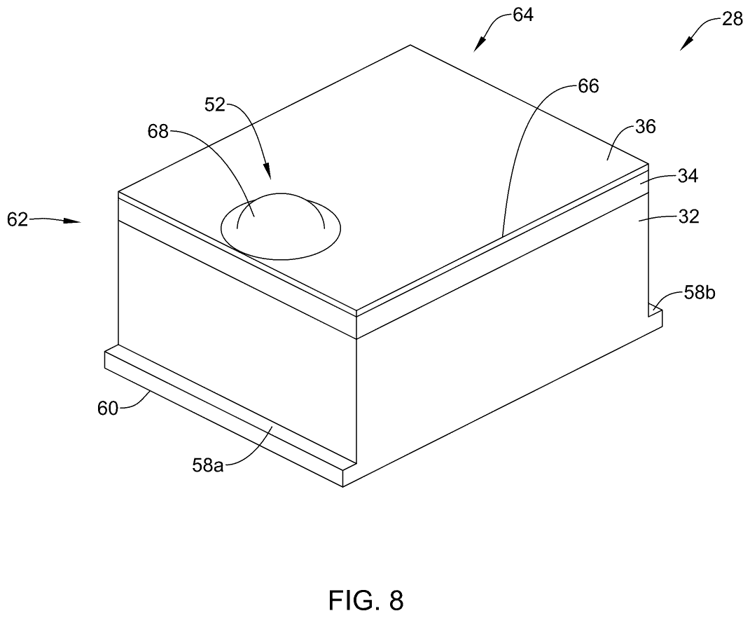

FIG. 8 is a perspective view of the illustrative transducer 28 in which a conductive deposit or pre-tin solder 68 has been applied or deposited to the upper surface 66 of the PZT layer 34 to create the connection point 52. The conductive deposit 68 or connection point 52 may have a surface area less than a surface area of an upper surface 33 of the PZT layer 34. The pre-tin solder 68 may be solder-jetting applied to the upper surface 66 of the PZT layer 34 before the matching layer 36 is applied. For example, the pre-tin solder 68 may be applied between dicing the PZT wafer/backing layer (step 116 of FIG. 3) and applying the matching layer (step 118 of FIG. 3). After the matching layer 36 is applied, the matching layer may be ablated off at the site of the pre-tin solder 68 such than an electrical connection may be made to the upper surface of the PZT layer 34 via the pre-tin solder 68. In some cases, the matching layer 36 may be knife-cast in a stencil over the PZT layer 34. The matching layer 36 may be ground, lapped, or otherwise machined down to a desired thickness (e.g., in the range of about 11-12 μm). During grinding, the pre-tin solder 68 may also be machined down (not explicitly shown in FIG. 8) such that the pre-tin solder 68 and the matching layer 36 have approximately the same height. In some examples, a thickness of the metallic layered coating on the PZT wafer 200 may be increased (relative to transducers 28 with no pre-tin solder 68) to improve the reliability of the solder bond of the pre-tin solder 68 with the PZT layer 34. In an alternative embodiment, a conductive matching layer may be knife cast over the PZT wafer 200/backing layer block. A metallic layered coating may be applied over the matching layer and the pre-tin solder disposed over the metallic layered coating.

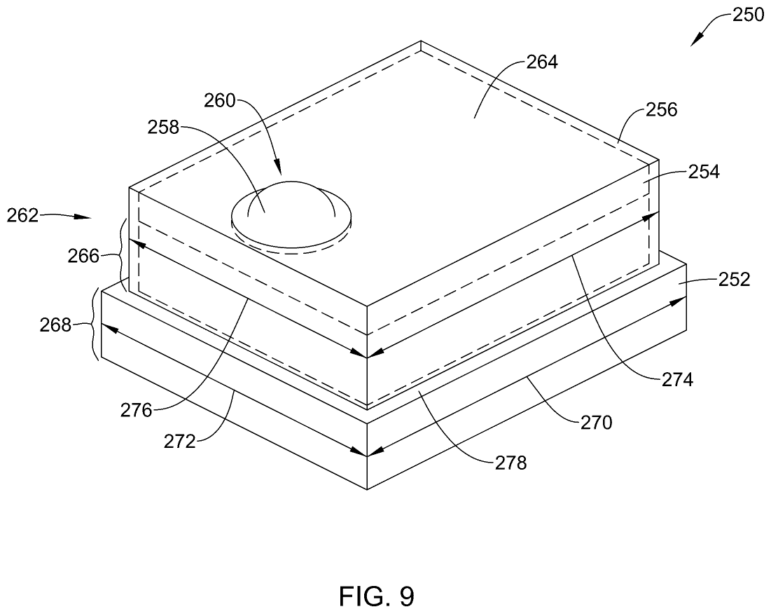

FIG. 9 is a perspective view of another illustrative transducer 250 which is formed by the process described with respect to FIG. 3. The transducer 250 includes a backing layer 252, a PZT layer 254, and a matching layer 256. The transducer 250 further includes a pre-tin solder 258 that has been applied to an electrical connection point 260 similar in form and function to the pre-tin solder 68 described with respect to FIG. 8. However, the pre-tin solder 258 may be omitted. The matching layer 256 has been removed or ablated to expose the connection point 260, as described with respect to FIG. 8. The connection point 260 may be adjacent to a proximal end 262 of the transducer 250. The matching layer 256 may extend across an upper surface 264 of the PZT layer 254 and along the lateral and axial sides of the PZT layer 254 and the lateral and axial sides of an upper portion 266 of the backing layer 252. The matching layer 256 may extend around an entire perimeter of the transducer 250 while a lower portion 268 of the lateral sides of the backing layer 252 remains free from contacting the matching layer 256.

The lower portion 268 of the backing layer 252 may have a first length 270 and a first width 272. The upper portion 266 of the backing layer 252 may have a second length 274 shorter than the first length 270 and a second width 276 shorter than the first width 272. The upper portion 266 may be centered relative to the lower portion 268 such that the lower portion 268 extends axially (e.g., parallel with a longitudinal axis of the transducer 250) and laterally beyond the upper portion 266 by a same distance around an entirety of the perimeter of the transducer 250 to define a ledge 278 extending around the perimeter of the transducer 250. The ledge 278 may be formed during the dicing step 116. During dicing of the PZT wafer/backing layer block, the cuts may not extend through an entire thickness of the backing layer in either the axially (columns) or lateral (rows) directions such that the columns and rows of the array 220 remain connected. When the matching layer 256 is applied, the matching layer 256 may extend along the lateral edges of the PZT wafer and along the backing layer until the bottom of the cut is reached. When the transducers are singulated, a dicing saw having a thinner than the dicing saw blade used for initial dicing may be used such that singulation cut is thinner than the original dicing cut. This may define the ledge 278 and leave a layer of matching layer 256 along the lateral sides of a portion of the transducer 250. It is contemplated that the ledge 278 may help provide better adhesion between the matching layer 256 and the backing layer 252. The increased adhesion may help resist peeling of the matching layer 256 during singulation (e.g., step 124).

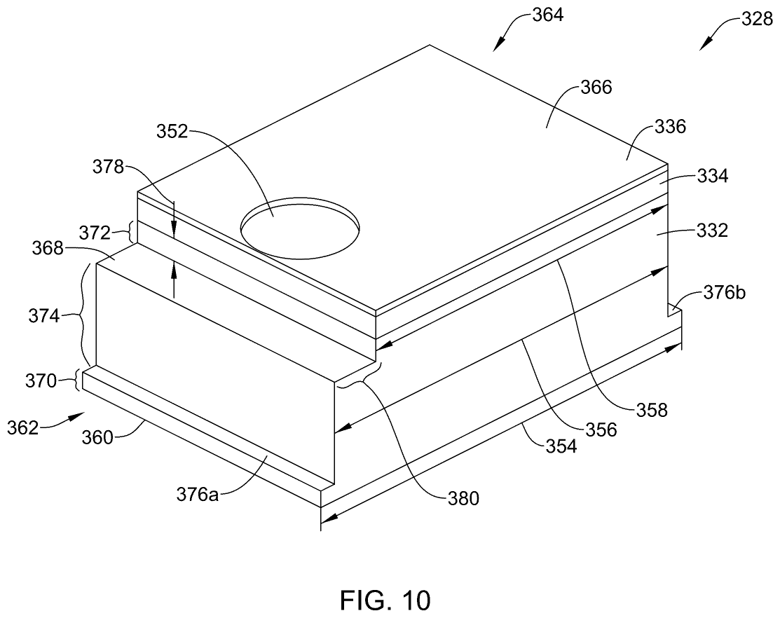

FIG. 10 is a perspective view of another illustrative transducer 328 extending from a proximal end 362 to a distal end 364 which is formed by the process described with respect to FIG. 3. The transducer 328 includes a backing layer 332, a PZT layer 334, and a matching layer 336. A region of the matching layer 336 has been removed or ablated to define a connection point 352. The connection point 352 may be adjacent to a proximal end 362 of the transducer 328. The matching layer 336 may extend across an upper surface 366 of the PZT layer 334. However, in some cases, the matching layer 336 may extend along the lateral sides of the PZT layer 334 and/or portions of or an entirety of the lateral sides of the backing layer 332.

The transducer 328 may include a lower portion 370 adjacent to a bottom 360 surface thereof, an upper portion 372 adjacent to the PZT layer 334, and an intermediate portion 374 therebetween. The lower portion 370 of the backing layer 332 may have a first length 354. The intermediate portion 374 may have a second length 356 less than the first length 354. The upper portion 372 may have a third length 358 less than the second length 356. Generally, the length of the backing layer 332 may increase from an upper surface adjacent to the PZT layer 334 to the lower or bottom surface 360 in an incremental or stepwise manner to define a first ledge 368, a second ledge or foot 376a, and a third ledge or foot 376b. The second and third feet 376a, 376b may extend approximately an equal distance from the proximal end of the intermediate portion 374 and a distal end of the intermediate portion 374. The thickness of the feet 376a, 376b may be determined during the initial dicing (step 116) The feet 376a, 376b may provide the electrical connection between adjacent transducers during the manufacturing process (e.g., when the transducers 328 are in an array such as array 220). The feet 376a, 376b may be created when a dicing saw is used for singulation which has a thinner blade than the dicing saw used for initial dicing (e.g., step 116 of FIG. 3). For example, less material may be removed during singulation with a thinner blade.

The first ledge 368 may also be manufactured during the initial dicing (e.g., step 116). For example, the initial dicing may include an additional dicing step in which the dicing blade is used to create a second cut extending in the lateral direction in which the second cut extends through less of the thickness of the PZT wafer/backing layer block than the first cut. This may remove a proximal portion of the PZT wafer and a thickness 378 and length 380 of the backing layer to define the first ledge 368. The first ledge 368 extends proximally from the proximal end 362 of the transducer 328. The first edge 368 may support a non-conductive potting adhesive which is used to secure the portions of a coaxial cable to the transducer 328 and/or housing 26. It is contemplated that the ledge 368 may reduce the volume of potting material required to secure the coaxial cable. Additional coupling mechanisms are described in commonly assigned patent application attorney docket number 2001.3796100 titled “IVUS Catheters” filed of even date herewith, which is hereby incorporated by reference. While not explicitly shown, in some examples, the matching layer 336 may extend over and cover the first ledge 368. It is contemplated that the first ledge 368 may be used for the negative connection for the electrical tests of step 122. If a matching layer 336 is disposed over the first ledge 368, the matching layer 336 may need to be removed or pierced to complete the electrical connection.

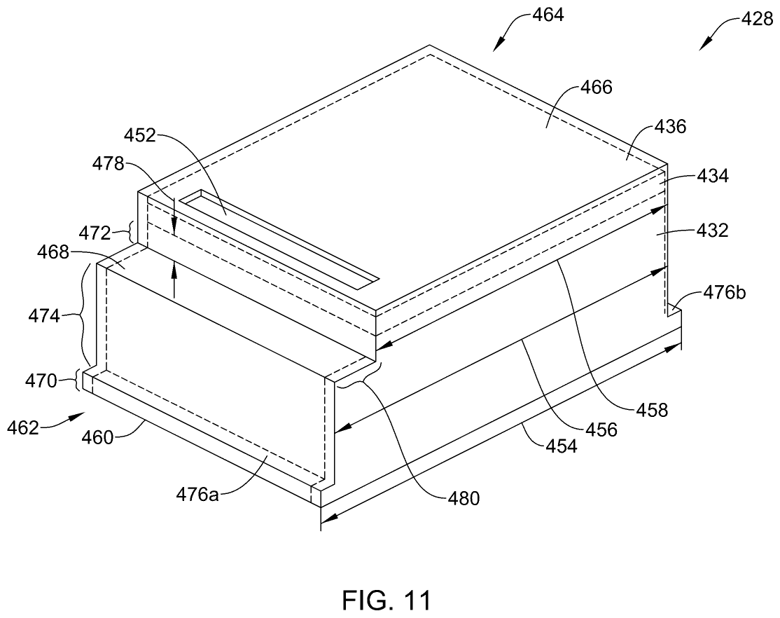

FIG. 11 is a perspective view of another illustrative transducer 428 extending from a proximal end 462 to a distal end 464 which is formed by the process described with respect to FIG. 3. The transducer 428 includes a backing layer 432, a PZT layer 434, and a matching layer 436. A region of the matching layer 436 has been removed or ablated to define a connection point 452. In FIG. 11, the connection point 452 may be generally rectangular. It is contemplated that a generally rectangular or strip-like connection point 452 may allow an electrically conductive adhesive or solder to be dispensed in a line along the proximal edge of the active portion of the transducer 428 (e.g., proximal edge of the PZT layer 434) to couple the PZT layer 434 with a coaxial cable. Additional coupling mechanisms are described in commonly assigned patent application attorney docket number 2001.3747100 titled “Electrical connections for IVUS Catheters” filed of even date herewith, which is hereby incorporated by reference. The connection point 452 may be adjacent to a proximal end 462 of the transducer 428. The matching layer 436 may extend across an upper surface 466 of the PZT layer 434 and along the lateral and axial (proximal and distal) sides of the PZT layer 434 and the lateral and axial (proximal and distal) sides of an entirety of a perimeter the backing layer 432. The lower or bottom surface 460 of the backing layer 432 may remain free from the matching layer 436. However, in some embodiments, the matching layer 436 may be disposed over less than an entirety of the lateral and/or axial (proximal and distal) surfaces of the backing layer 432.

The transducer 428 may include a lower portion 470 adjacent to a bottom 460 surface thereof, an upper portion 472 adjacent to the PZT layer 434, and an intermediate portion 474 therebetween. The lower portion 470 of the backing layer 432 may have a first length 454. The intermediate portion 474 may have a second length 456 less than the first length 454. The upper portion 472 may have a third length 458 less than the second length 456. Generally, the length of the backing layer 432 may increase from an upper surface adjacent to the PZT layer 434 to the lower or bottom surface 460 in an incremental or stepwise manner to define a first ledge 468, a second ledge or foot 476a, and a third ledge or foot 476b. The second and third feet 476a, 476b may extend approximately an equal distance from the proximal end of the intermediate portion 474 and a distal end of the intermediate portion 474. The thickness of the feet 476a, 476b may be determined during the initial dicing (step 116) The feet 476a, 476b may provide the electrical connection between adjacent transducers during the manufacturing process (e.g., when the transducers 428 are in an array such as array 220). The feet 476a, 476b may be created when a dicing saw is used for singulation which has a thinner blade than the dicing saw used for initial dicing (e.g., step 116 of FIG. 3). For example, less material may be removed during singulation with a thinner blade.

The first ledge 468 may also be manufactured during the initial dicing (e.g., step 116). For example, the initial dicing may include an additional dicing step in which the dicing blade is used to create a second cut extending in the lateral direction in which the second cut extends through less of the thickness of the PZT wafer/backing layer block than the first cut. This may remove a proximal portion of the PZT wafer and a thickness 478 and length 480 of the backing layer to define the first ledge 468. The first ledge 468 extends proximally from the proximal end 462 of the transducer 428. The first ledge 468 may support a non-conductive potting adhesive which is used to secure the portions of a coaxial cable to the transducer 428 and/or housing 26. It is contemplated that the ledge 468 may reduce the volume of potting material required to secure the coaxial cable. Additional coupling mechanisms are described in commonly assigned patent application attorney docket number 2001.3796100 titled “IVUS Catheters” filed of even date herewith. While not explicitly shown, in some examples, the matching layer 436 may extend over and cover the first ledge 468. It is contemplated that the first ledge 468 may be used for the negative connection for the electrical tests of step 122. If a matching layer 436 is disposed over the first ledge 468, the matching layer 436 may need to be removed or pierced to complete the electrical connection.

The materials that can be used for the various components of the system 10 (and/or other systems disclosed herein) may include those commonly associated with medical devices. For simplicity purposes, the following discussion makes reference to the shaft 12 and other components of the system 10. However, this is not intended to limit the devices and methods described herein, as the discussion may be applied to other similar tubular members and/or components of tubular members or devices disclosed herein.

The shaft 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some cases, the shaft 12 and/or other components of the system 10 may include polymeric coatings for fillers. Some examples for suitable polymers for coating or fillers may include, but are not limited to, parylene (poly-para-xylylene), poly-dimethyl siloxane (PDMS), poly-methyl methacrylate (PMMA), and poly-(vinylidene fluoride) (PVDF), polyacrylonitrile (PAN), epoxy resins, or the like.

Adhesives or electrically conductive adhesives may be used in the coupling of various components of the shaft and/or other components of the system 10. Electrically conductive adhesives may include a conductive component, such as, but not limited to iron, silver, copper, nickel, graphite, or the like, suspended in an adhesive. Some examples of adhesives include acrylics, epoxies, urethanes, hydrocolloids, hydrogels, cyanoacrylates, silicones, or the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method of manufacturing an ultrasound transducer for use in an intravascular ultrasound catheter, the method comprising:
forming a composite PZT structure;
applying a backing layer to the composite PZT structure;
applying a metallic layered coating to the backing layer;
dicing the composite PZT structure and the backing layer to form an array of transducer elements, wherein two or more transducer elements remain mechanically connected at the backing layer after dicing;
applying a matching layer to the diced composite PZT structure; and
creating a connection point through the matching layer.

2. The method of claim 1, wherein dicing the composite PZT structure and the backing layer comprises:
performing a first dicing operation to separate the array of transducer elements into columns; and
performing a second dicing operation to partially separate the array of transducer elements into rows while maintaining electrical connectivity between transducer elements through the backing layer.

3. The method of claim 1, wherein applying the backing layer comprises:
casting a conductive epoxy onto the composite PZT structure
curing the conductive epoxy; and
grinding the cured conductive epoxy to a specified thickness.

4. The method of claim 1, further comprising testing the transducer elements while they remain connected in the array of transducer elements.

5. The method of claim 4, wherein testing the transducer elements comprises: coupling a negative lead to a backing layer of a first transducer element in a column of the array of transducer elements;
coupling a positive lead to a connection point on the first transducer element;
measuring an electrical property of the first transducer element;
maintaining the coupling of the negative lead to the backing layer; and
sequentially coupling the positive lead to connection points on subsequent transducer elements in the column to measure the electrical property of each transducer element in the column.

6. The method of claim 1, further comprising singulating the transducer elements.

7. The method of claim 1, wherein forming the composite PZT structure comprises:
dicing a first PZT wafer and a second PZT wafer;
applying a thin layer of a polymer coating to the diced surfaces of the PZT wafers; and
pressing the first and the second diced PZT wafers together to form the composite PZT structure.

8. The method of claim 7, wherein after dicing the first PZT wafer and the second PZT wafer each of the first and second PZT wafers include a plurality of pillars.

9. The method of claim 8, wherein each pillar of the plurality of pillars has a width to height ratio in the range of 0.15 to about 0.35.

10. The method of claim 1, wherein applying a matching layer to the diced composite PZT structure comprises:
depositing a conductive deposit on the diced composite PZT structure; and
applying the matching layer over the conductive deposit and the diced composite PZT structure; and
wherein creating a connection point through the matching layer comprises:
removing a portion of the matching layer to expose the conductive deposit.

11. An ultrasound transducer for an intravascular ultrasound catheter, comprising:
a backing layer having a lower portion and an upper portion;
a piezoelectric layer disposed on the upper portion of the backing layer;
a matching layer disposed on the piezoelectric layer; and
a connection point formed through the matching layer;
wherein the lower portion of the backing layer has a first length greater than a second length of the upper portion of the backing layer.

12. The ultrasound transducer of claim 11, wherein the lower portion of the backing layer extends beyond the upper portion to form a ledge.

13. The ultrasound transducer of claim 12, wherein the ledge extends around an entire perimeter of the backing layer.

14. The ultrasound transducer of claim 11, further comprising a pre-tin solder disposed on the piezoelectric layer at the connection point.

15. The ultrasound transducer of claim 11, further comprising a metallic layered coating on a bottom surface of the backing layer.

16. An ultrasound transducer for an intravascular ultrasound catheter comprising:
a backing layer having a lower portion, an intermediate portion, and an upper portion;
a piezoelectric layer disposed on the upper portion of the backing layer;
a matching layer disposed on the piezoelectric layer; and
a connection point formed through the matching layer;
wherein the lower portion of the backing layer has a first length, the intermediate portion has a second length less than the first length, and the upper portion has a third length less than the second length.

17. The ultrasound transducer of claim 16, wherein the intermediate portion of the backing layer extends proximally beyond a proximal end of the upper portion to form a first ledge.

18. The ultrasound transducer of claim 17, wherein the first ledge extends proximally to a proximal end of the piezoelectric layer.

19. The ultrasound transducer of claim 17, wherein the lower portion of the backing layer extends proximally beyond a proximal end of the intermediate portion to form a second ledge.

20. The ultrasound transducer of claim 19, wherein the lower portion of the backing layer extends distally beyond a distal end of the intermediate portion to form a third ledge.
